(19) United States
(12) Patent Application Publication
(10) Pub. No.: US 2026/0345613 A1
(43) Pub. Date: Nov. 5, 2026

(54) Compositions and Methods for Reducing Toxic Effects of Alcohol Consumption

(71) Applicant: Jason Alan Ostendorf, Perry Hall, MD (US)

(72) Inventor: Jason Alan Ostendorf, Perry Hall, MD (US)

(21) Appl. No.: 19/198,082

(22) Filed: May 4, 2025

(51) Int. Cl. A61K 31/519; A61K 31/198; A61K 31/51; A61K 33/06

(52) U.S. Cl. CPC A61K 31/519; A61K 31/198; A61K 31/51; A61K 33/06

(57) ABSTRACT

A composition and method for reducing the toxic effects of alcohol consumption in humans. The composition comprises L-cysteine, N-acetylcysteine, thiamine, folate, and magnesium citrate, optionally combined with an aldehyde dehydrogenase (ALDH) enhancer. The formulation is administered orally approximately one hour prior to alcohol ingestion and is configured to reduce acetaldehyde accumulation, oxidative stress, and nutrient depletion associated with ethanol metabolism. L-cysteine binds acetaldehyde directly, while N-acetylcysteine supports glutathione synthesis to mitigate reactive oxygen species. Thiamine and folate replenish key micronutrients often depleted by alcohol, and magnesium citrate restores electrolyte balance. The composition is designed to preserve the psychoactive effects of alcohol, including euphoria and relaxation, while limiting adverse physiological and neurological consequences. The invention provides an acute-use supplement to reduce harm during social or heavy drinking without requiring chronic administration or altering the subjective experience of alcohol intoxication.

### FIELD OF THE INVENTION

The present invention relates generally to nutritional and pharmacological compositions. More specifically, it relates to a composition formulated to reduce the toxic effects of alcohol consumption in humans by mitigating acetaldehyde toxicity, oxidative stress, and nutrient depletion without suppressing the desired psychoactive effects of alcohol.

### BACKGROUND OF THE INVENTION

Alcohol (ethanol) consumption is associated with a variety of toxic effects resulting from its metabolism, particularly the accumulation of acetaldehyde, oxidative damage to cells, and the depletion of key micronutrients. Acetaldehyde is a highly reactive and toxic metabolite produced during the breakdown of ethanol, primarily by alcohol dehydrogenase (ADH) in the liver. Inadequate clearance of acetaldehyde, due to limited aldehyde dehydrogenase (ALDH) activity, leads to symptoms of toxicity and long-term damage to hepatic and neurological systems.

Oxidative stress arises from reactive oxygen species (ROS) generated by ethanol metabolism via cytochrome P450 2E1 (CYP2E1) and contributes to inflammation and cellular injury. Furthermore, ethanol disrupts the absorption and retention of several key nutrients including thiamine (vitamin B1), folate (vitamin B9), and magnesium, impairing neurological and metabolic function.

Current approaches to mitigating alcohol-induced damage are limited in effectiveness or require chronic administration. There remains a need for an acute-use composition, taken shortly before alcohol consumption, that offers biochemical protection without diminishing the positive subjective effects of ethanol intoxication.

### SUMMARY OF THE INVENTION

The present invention provides a composition and method for reducing the harmful effects of alcohol consumption, particularly acetaldehyde toxicity, oxidative stress, and nutrient depletion, while preserving the psychoactive effects of ethanol. The composition is intended to be taken orally approximately one hour prior to the ingestion of alcohol.

The formulation comprises L-cysteine, N-acetylcysteine (NAC), thiamine, folate, and magnesium citrate, optionally combined with an aldehyde dehydrogenase (ALDH) activity enhancer. Each component contributes to specific protective mechanisms associated with alcohol metabolism.

The invention also provides methods of use of the composition to reduce the incidence and severity of alcohol-related toxic symptoms, such as headache, nausea, cognitive impairment, and hepatic stress, particularly during episodes of acute heavy drinking.

### DETAILED DESCRIPTION OF THE INVENTION

### Formulation Components

L-Cysteine (600 mg): L-cysteine is a sulfur-containing amino acid that directly binds acetaldehyde, forming stable and non-toxic conjugates such as 2-methylthiazolidine-4-carboxylic acid. This binding reduces systemic exposure to acetaldehyde and protects tissues from its toxic effects. The selected dose balances effectiveness with gastrointestinal tolerability.

N-Acetylcysteine (600 mg): NAC is a prodrug for L-cysteine and a known precursor for the synthesis of glutathione, a critical antioxidant that neutralizes ROS generated during ethanol metabolism. NAC has a well-established safety profile and is effective in mitigating oxidative damage in hepatic tissue.

Thiamine (100 mg): Thiamine (vitamin B1) is an essential coenzyme for carbohydrate metabolism and neuronal function. Alcohol consumption significantly depletes thiamine levels, increasing the risk of lactic acidosis and Wernicke-Korsakoff syndrome. High-dose supplementation prevents acute deficiency during alcohol intake.

Folate (400 mcg): Folate (vitamin B9) is involved in DNA synthesis, repair, and methylation. Ethanol impairs folate absorption and increases its excretion. The included dose replenishes folate stores and reduces systemic inflammation associated with alcohol metabolism.

Magnesium Citrate (150 mg elemental magnesium): Magnesium is a critical electrolyte involved in enzymatic processes, neuronal signaling, and cardiovascular stability. Ethanol increases urinary magnesium excretion, contributing to neuromuscular irritability and arrhythmias. Magnesium citrate provides bioavailable magnesium to support systemic stability.

(Optional) ALDH Enhancer (50 mg): The composition optionally includes an aldehyde dehydrogenase enhancer such as Alda-1 or a similar compound. This agent is designed to increase the catalytic efficiency of ALDH2, enhancing the conversion of acetaldehyde to non-toxic acetate and reducing peak acetaldehyde levels. While not currently approved for human use, inclusion of such an agent is contemplated as part of future formulations.

### Dosage and Administration

The composition is administered orally, preferably in capsule or tablet form, in a single dose of approximately 1,900 mg of active ingredients. It is taken approximately one hour prior to alcohol consumption, allowing sufficient time for absorption and activation of the protective mechanisms.

### Method of Use

A method for reducing alcohol-induced toxicity in a subject comprises administering to the subject a composition comprising effective doses of L-cysteine, N-acetylcysteine, thiamine, folate, and magnesium citrate, optionally with an ALDH enhancer, wherein the composition is administered orally within 30 to 90 minutes before alcohol consumption.

### Alternative Embodiments

In alternative embodiments, the composition may be provided in powder, liquid, effervescent, or chewable form. Dosage ranges may vary depending on body weight, liver function, or anticipated alcohol consumption. For example:

L-cysteine may range from 400-1200 mg

NAC from 300-1200 mg

Thiamine from 50-200 mg

Folate from 400-800 mcg

Magnesium (elemental) from 100-300 mg

The composition may also be co-formulated with flavoring agents, binders, or enteric coatings to improve bioavailability and user compliance.

### INDUSTRIAL APPLICABILITY

The invention is useful in consumer health products, over-the-counter supplements, and potentially pharmaceutical applications aimed at reducing the toxic effects of alcohol consumption. It has applicability in social, occupational, and medical contexts where individuals may consume moderate to high amounts of ethanol.

1. A composition for reducing alcohol-induced toxicity in a human subject, the composition comprising:
L-cysteine in an amount between 400 mg and 1200 mg;
N-acetylcysteine in an amount between 300 mg and 1200 mg;
thiamine in an amount between 50 mg and 200 mg;
folate in an amount between 400 mcg and 800 mcg; and
magnesium citrate providing between 100 mg and 300 mg of elemental magnesium;
wherein the composition is formulated for oral administration approximately one hour prior to alcohol consumption and is configured to reduce the toxic effects of ethanol metabolism, including acetaldehyde accumulation and oxidative stress, without inhibiting the psychoactive effects of alcohol.

2. The composition of claim 1, wherein the L-cysteine and N-acetylcysteine act synergistically to reduce acetaldehyde toxicity and support glutathione synthesis.

3. The composition of claim 1, further comprising an aldehyde dehydrogenase (ALDH) activity enhancer in an amount effective to increase the catalytic conversion of acetaldehyde to acetate.

4. The composition of claim 1, wherein the composition is provided in capsule or tablet form.

5. The composition of claim 1, wherein the composition is provided in a single oral dose of between 1500 mg and 2500 mg of total active ingredients.

6. The composition of claim 1, wherein the thiamine is in the form of thiamine mononitrate or thiamine hydrochloride.

7. The composition of claim 1, wherein the folate is in the form of folic acid or methylfolate.

8. The composition of claim 1, wherein the magnesium citrate is selected to optimize gastrointestinal tolerance and bioavailability.

9. The composition of claim 1, wherein the composition reduces symptoms associated with acute alcohol consumption, including headache, nausea, and cognitive impairment.
